(19) United States
(12) Patent Application Publication
(10) Pub. No.: US 2024/0131112 A1
(43) Pub. Date: Apr. 25, 2024

(54) USE OF METHANOBACTIN FOR TREATMENT OF IRON-RELATED DISEASES

(71) Applicants: Helmholtz Zentrum Muenchen - Deutsches Forschungszentrum für Gesundheit und Umwelt (GmbH), Neuherberg (DE); The Regents of the University of Michigan, Ann Arbor, MI (US); Iowa State University Research Foundation, Inc., Ames, IA (US)

(72) Inventors: Hans ZISCHKA, Munich (DE); Alan Angelo DISPIRITO, Ames, IA (US); Jeremy David SEMRAU, Ann Arbor, MI (US)

(21) Appl. No.: 18/274,981

(22) PCT Filed: Feb. 1, 2022
(86) PCT No.: PCT/EP2022/052263

§ 371 Date: Jul. 28, 2023

Related U.S. Application Data
(60) Provisional application No. 63/144,338, filed on Feb. 1, 2021.

(30) Foreign Application Priority Data
Feb. 3, 2021 (LU) 102472

(51) Int. Cl. A61K 38/16

(52) U.S. Cl. CPC A61K 38/164

(57) ABSTRACT

The present invention relates to a methanobactin reducing Fe3+ ions to Fe2+ ions for use in medicine and a pharmaceutical composition comprising said methanobactin as well as to a process for reducing Fe3+ ions to Fe2+ ions ex vivo.

### TECHNICAL FIELD OF THE INVENTION

The present invention relates to a methanobactin reducing Fe³⁺ ions to Fe²⁺ ions for use in medicine and a pharmaceutical composition comprising said methanobactin as well as to a method for reducing Fe³⁺ ions to Fe²⁺.

### BACKGROUND ART

Iron overload can occur among patients with hereditary hemochromatosis, thalassemia, sickle cell disease, aplastic anemia, myelodysplasia, and other diseases. Hereditary hemochromatosis is due to mutations in genes encoding proteins involved in limiting systemic iron uptake. Around 10% of the population are heterozygous carriers and 0.3-0.5% are homozygous. Currently, these diseases, which are caused by dysregulated iron uptake, are treated by regular bleeding (up to 500 ml/per week!) or iron chelators like deferiprone (Ferriprox), deferasirox (Exjade, Novartis net sales 2019: 995 Mio $, https://www.novartis.com/investors/financial-data/product-sales) and deferoxamine (Desferal). These chelators have unwanted side effects and are less effective than bleeding. However, they are only partially able to dissolve iron deposits.

Moreover, brain iron accumulation is associated with several neurodegenerative diseases such as Alzheimers disease, Parkinson disease, Dementia, Huntington disease (Liu et al, Front Neurosci. 2018; 12: 632; Agraval et al., Free Radical Biology and Medicine 2018, 120, 317-329; Moon et al., J Alzheimers Disease 2016, 51(3), 737-45), and multiple sclerosis (Stephenson et al., Nature Reviews Neurology, 2014, volume 10, 459-468).

In particular, iron and iron accumulation plays a role in senescence (Masaldan et al. Redox Biol, 2018,14:100-115), ageing (Timmers et al., NATURE COMMUNICATIONS| (2020), 11, 3570), ferroptotic cell death (Li et al. Cell Death & Disease, 11, Article number: 88), alcoholic liver disease (Kowdley, Gastroenterol Hepatol (N Y), 2016, 12(11): 695-698) or amyotrophic lateral sclerosis (Gajowiak et al., Postepy Hig Med Dosw (Online), 2016 Jun 30; 70(0):709-21).

Thus, it could be very beneficial to identify compounds which are able to help in the depletion of iron, in particular in dissolving iron deposits.

### SUMMARY OF THE INVENTION

The invention is directed to a methanobactin reducing Fe³⁺ ions to Fe²⁺ ions for use in medicine.

Further, the invention is directed to pharmaceutical composition comprising the methanobactin.

Furthermore, the invention is directed to a process for reduction of Fe³⁺ to Fe²⁺ ex vivo.

As shown in the examples, it has been surprisingly found that certain methanobactins are able to complex Fe³⁺ ions and reduce them to Fe²⁺. Most of excess iron is usually stored as Fe³⁺ by ferretin in animals, plants and bacteria. Thus, excess iron Fe³⁺ ions can be removed by the found methanobactins of the present invention. Therefore, the methanobactins of the present invention are useful in therapy of diseases caused by an accumulation of iron ions in the body.

### BRIEF DESCRIPTION OF THE FIGURES

FIG. 1: Ferric Iron Binding and Reduction to Ferrous Iron by MB-SB2

A. UV-visible absorption spectra of 50 nmol ml⁻¹ SB2-MB as isolated and following 5 nmol additions of FeCl₃. B. Absorbance of the oxazolone (O) and imidazolone (Δ) groups at 336 and 387 nm, respectively as a function of FeCl₃ to MB-SB2 molar ratios.

FIG. 2. Iron reductase activity of MB-SB2 but not by MB-OB3b

Absorption change at 562 nm of reaction mixtures containing 1 mM ferrozine plus 10 mM FeCl₃ ( [symbol] ), 1 mM ferrozine plus 23.4 μM MB-SB ( [symbol] ), 1 mM ferrozine plus 10 mM FeCl₃ and either 5.8 ( [symbol] ), 11.6 ( [symbol] ), 17.4 ( [symbol] ) or 23.4 ( [symbol] ) μM MB-SB2 (A) or MB-OB3b (B). C. Aqueous 4M FeCl₃ solution (a) and a 4M FeCl₃ solution plus 20 mM MB-SB2 4 hours after the addition of MB-SB2.

FIG. 3: Biliary iron excretion by MB-SB2 but not by MB-OB3

A. Biliary iron excretion. Upon liver perfusion, MB-SB2 brings iron to the bile whereas MB-OB3b does not. B. Fecal iron excretion. Upon intraperitoneal injection of MB-SB2 LPP Atp7b^{-/-} rats excrete iron in feces but not upon MB-OB3b injection. Dashed line indicates average fecal iron excretion of untreated rats.

FIG. 4: Water oxidation coupled to Fe(III) reduction by MB-SB2

Mass spectra of head space gas of a reaction mixture containing 2 mM MB-SB2 in 97% H₂ ¹⁸O following the addition of 20 mM FeCl₃.

FIG. 5: Control Huh7 cells (human hepatoma cell line) were preloaded for 24 hours with 50 μM FAC. The cells were then treated with 0.5 mM MB-SB2, 0.5 mM MB-OB3b and 0.5 mM DFO for 24 hours. In contrast to OB3B and DFO, MB-SB2 significantly reduces cellular iron concentration in iron-preloaded Huh7 cells to the untreated level. Statistical analysis was done by One-way ANOVA (N=4).

FAC: Iron ammonium citrate

UT : untreated Huh7 cells

Control: cells treated with 50 μM FAC for 24 h

DFO: Deferoxamine

### DETAILED DESCRIPTION OF THE INVENTION

The solution of the present invention is described in the following, exemplified in the appended examples, illustrated in the Figures and reflected in the claims.□

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

The invention is directed to a methanobactin reducing Fe³⁺ ions to Fe²⁺ ions for use in medicine.

The term “methanobactin” as used herein in particular encompasses modified peptides characterized by the presence of one oxazolone ring and a second oxazolone, imidazolone or pyrazinedione ring. The two rings are separated by 2-5 amino acid residues. Each ring has an adjacent thioamide group.

Preferably, the methanobactin is MB-SB2 and comprises a primary structure according to formula (I). More preferably, the methanobactin has a primary structure according to formula (I) and is MB-SB2.

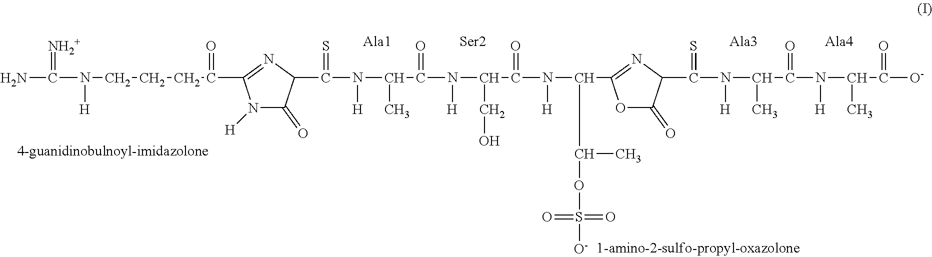 [SMILES: CC(NC(=O)C(C)NC(=S)C1N=C(C(NC(=O)C(CO)NC(=O)C(C)NC(=S)C2N=C(C(=O)CCCNC(N)=[NH2+])NC2=O)C(C)OS(=O)(=O)[O-])OC1=O)C(=O)[O-]]

When complexing Fe²⁺ or Fe³⁺, the resulting methanobactin complexes are envisioned to have a formula according to formula (II). Thus, reducing Fe³⁺ to Fe²⁺ is envisioned to comprise an intermediate structure according to formula (II).

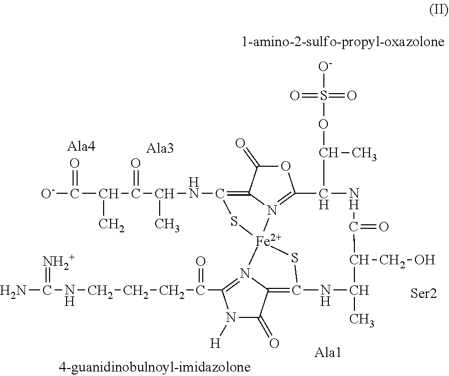

When used herein, the terms “complexing” and “binding” are used interchangeably, i.e. for instance a methanobactin “binding” iron is to be understood as a methanobactin “complexing” iron, and vice versa. The term “complexing” generally means forming a complex consisting of a central ion and surrounding array of molecules that are known as ligands or complexing agents. For the present invention, the central ion will be iron (i.e. Fe²⁺ or Fe³⁺), and the ligand will be methanobactin. One methanobactin will typically complex one iron ion, forming a methanobactin-iron complex, respectively.

In the presence of excess Fe³⁺, the methanobactin reduces Fe³⁺ to Fe²⁺ preferably at a rate of 0.1 to 200, more preferably of 0.5 to 5, most preferably of 0.7 to 3, particularly preferred at a rate of 1 Fe³⁺ reduced per minute per methanobactin.

Preferably, the reduction is carried out catalytically, with the methanobactin as catalyst. In the present invention, the term “catalytically” is defined as a reaction wherein a “catalyst”—molecule increases the rate of reaction speed of a reaction, by forming intermediates, ideally in such a manner that a technically useful overall reaction speed is achieved. Within the present invention, the “catalyst” is regenerated after forming the intermediates, releasing the regenerated catalyst and the intended reaction products according to formula (III).

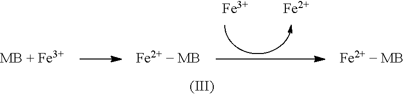

The methanobactin of the present invention may utilize a variety of electron donors for Fe³⁺ reduction. Preferably, the reducing agent is H₂O, generating molecular oxygen during the reduction process.

It is envisioned that the reduction preferably takes place according to the following equation:

4FeCl₃+Methanobactin+2H₂O→3Fe²⁺+Fe(II)-Methanobactin+12Cl⁻+4H⁺+O₂.

The person skilled in the art will readily understand that methanobactin-iron complexes will typically form after administration of the methanobactin to the subject, when methanobactin complexes and thereby depletes (excess) iron in the subject's body.

The term “methanobactin” includes naturally occurring methanobactins and functional variants, fragments and derivatives thereof which retain the capability of complexing iron (i.e., Fe²⁺ and Fe³⁺), and preferably bind Fe³⁺ with a binding affinity that is comparable or even higher than that of the naturally occurring methanobactins.

The term “methanobactin variant” refers to methanobactins having the general methanobactin formula of a “parent” methanobactin, but containing at least one amino acid substitution, deletion, or insertion as compared to the parent methanobactin, provided that the variant retains the desired iron-binding affinity and/or biological activities described herein.

“Methanobactin derivatives” are chemically modified methanobactins. Generally, all kind of modifications are comprised by the present invention as long as they do not abolish the beneficial effects of the methanobactins. That is, methanobactin derivatives preferably retain the iron-binding affinity and/or biological activity of the methanobactins they are derived from. Methanobactin derivatives also include stabilized methanobactins as described in the following.

Possible chemical modifications in the context of the present invention include acylation, acetylation or amidation of the amino acid residues. Other suitable modifications include, e.g., extension of an amino group with polymer chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e.g., methylation, propylation, butylation), arylation, and etherification may be possible and are also envisaged. Further chemical modifications envisaged herein are ubiquitination, conjugation to therapeutic or diagnostic agents, labeling (e.g., with radionuclides or various enzymes), and insertion or substitution by chemical synthesis of non-natural amino acids.

Other possible modifications may involve removal of the sulfate group and/or replacement of oxazolone group with the more stable imidazolone or pyrazinedione group. Gene additions and/or deletions of genes from the operons of Group II methanobactins into Group I or vice versa should result in alteration may result in a change in the type of ring (note: Group I and II methanobactins are described in Semrau et al. 2020.FEMS Microbiol Lett. 367: fn045). Replacement of oxazolone group(s) with either imidazolone or pyrazinedione group(s) should increase the stability of methanobactin to the point where oral administration may be possible.

For the purpose of the invention the methanobactin as defined above also includes the pharmaceutically acceptable salt(s) thereof. The phrase “pharmaceutically acceptable salt(s)”, as used herein, means those salts of methanobactins that are safe and effective for treatment. Pharmaceutically acceptable salts include those formed with anions such as those derived from hydrochloric, phosphoric, acetic, oxalic, tartaric acids, choline etc., and those formed with cations such as those derived from sodium, potassium, ammonium, calcium, ferric hydroxides, isopropylamine, triethylamine, 2-ethylamino ethanol, histidine, procaine, etc.

As set forth previously, the methanobactin fragments, variants and derivatives preferably retain the advantageous capabilities of the methanobactins evaluated in the appended examples.

The methanobactin may be derived from Methylocystis sp. strain SB2.

As discussed above and as shown in example 2, the methanobactin according to the invention is able to remove excess iron Fe³⁺ ions by reducing them to Fe²⁺ and complexing them in form of the respective Fe²⁺ ions. Therefore, the methanobactins of the present invention are useful in therapy of diseases caused by an accumulation of iron ions in the body and formation of iron deposists. The following diseases are related to iron accumulation, as also mentioned in the introduction, and thus their therapy benefits from agents which help to deplete iron deposits such as the methanobactin of the present invention.

Further, the invention is related to the methanobactin for use in the treatment of an iron-overload disorder, neurodegenerative diseases, iron overload due to red blood cell transfusions, senescence, ageing, ferroptotic cell death, alcoholic liver disease or amyotrophic lateral sclerosis.

The iron-overload disorder may be caused by a disease which is selected from the group consisting of hereditary hemochromatosis, thalassemia, sickle cell disease, aplastic anemia, myelodysplasia.

The neurogenerative diseases may be selected from the group consisting of Alzheimers disease, Parkinson disease, Dementia, Huntington disease, and multiple sclerosis.

A better understanding of the present invention and of its advantages will be had from the following examples, offered for illustrative purposes only. The examples are not intended to limit the scope of the present invention in any way.

The invention is further directed to a pharmaceutical composition comprising the methanobactin as described above for use in medicine, particular for the indications described above.

As set out in the foregoing, a pharmaceutical composition comprising methanobactin, is also envisaged herein. Accordingly, further aspects of the invention include a pharmaceutical composition comprising methanobactin as described herein and the use of said methanobactin for the manufacture of a pharmaceutical composition. The term “pharmaceutical composition” particularly refers to a composition suitable for administering to a human. However, compositions suitable for administration to non-human animals are also envisaged herein.

The pharmaceutical composition and its components (i.e. active ingredients and optionally excipients or carriers) are preferably pharmaceutically acceptable, i.e. capable of eliciting the desired therapeutic effect without causing undesirable or at least acceptable local or systemic effects. Pharmaceutically acceptable compositions of the invention may in particular be sterile and/or pharmaceutically inert. Specifically, the term “pharmaceutically acceptable” may mean approved by a regulatory agency or other generally recognized pharmacopoeia for use in animals, and more particularly in humans.

The methanobactin described herein is preferably present in the pharmaceutical composition in a therapeutically effective amount. By “therapeutically effective amount” is meant an amount of methanobactin that elicits the desired therapeutic effect. The exact amount dose will depend on the purpose of the treatment, and will be ascertainable by one skilled in the art using known techniques. Therapeutic efficacy and toxicity can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are generally preferred.

The pharmaceutical composition is envisaged to comprise a methanobactin as described herein, particularly in stabilized form, and preferably in a therapeutically effective amount, optionally together with one or more carriers, excipients and/or additional active agents.

“Excipients” include fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. Exemplary suitable carriers for use in the pharmaceutical composition of the invention include saline, buffered saline, dextrose, and water.

The pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. Processes known per se for producing medicaments are indicated in Forth, Henschler, Rummel (1996) Allgemeine und spezielle Pharmakologie und Toxikologie, Urban & Fischer.

A variety of routes are conceivable for administration of the methanobactins and pharmaceutical compositions according to the present invention. Typically, administration will be accomplished parentally, but oral administration is also envisaged. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration. Preferably, administration is accomplished intraperitoneally and intraveneously.

The invention is further directed to a process for reduction of Fe³⁺ to Fe²⁺ ex vivo, comprising, contacting the methanobactin as defined above with Fe³⁺ ions in a solution optionally in the presence of a suitable reduction agent.

Preferably, the methanobactin is a catalyst and a reduction agent is present.

The reduction agent may be Nicotinamidadenindinukleotid (NADH) or water.

The solvent may be a polar protic or aprotic solvent.

Preferably, the solvent and/or reduction agent is water.

Preferably, the reduction takes place at a rate of 0.5 to 5, more preferably 0.7 to 3, most preferably 1 Fe³⁺ reduced per minute per methanobactin.

Preferably, molecular oxygen is generated during the process.

### EXAMPLES OF THE INVENTION

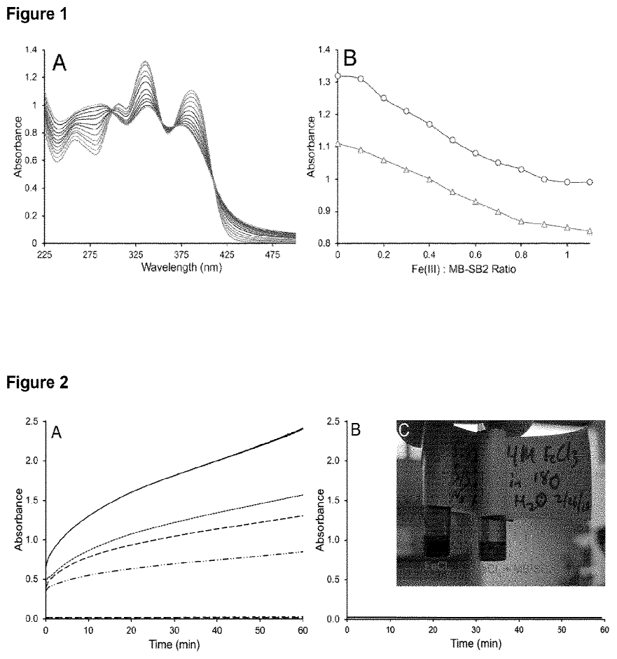

### Example 1: Ferric Iron Binding and Reduction to Ferrous Iron by MB-SB2 (FIG. 1)

The UV-visible absorption spectra of 50 nmol ml⁻¹ SB2-MB as isolated and following 5 nmol additions of FeCl₃ has been measured.

### Example 2: Iron Reduction (FIG. 2)

Ferrozine assay was used to determine iron reductase activity (1, 2).
1. Carter P. 1971. Spectrometric determination of serum iron at the submicrogram level with a new reagent (ferrozine). Anal Biochem 40:450-458.
2. Moody M D, Dailey H A. 1983. Aerobic ferrisiderophore reductase assay and activity stain for native polyacrylamide gels. Anal Biochem 134:235-239.

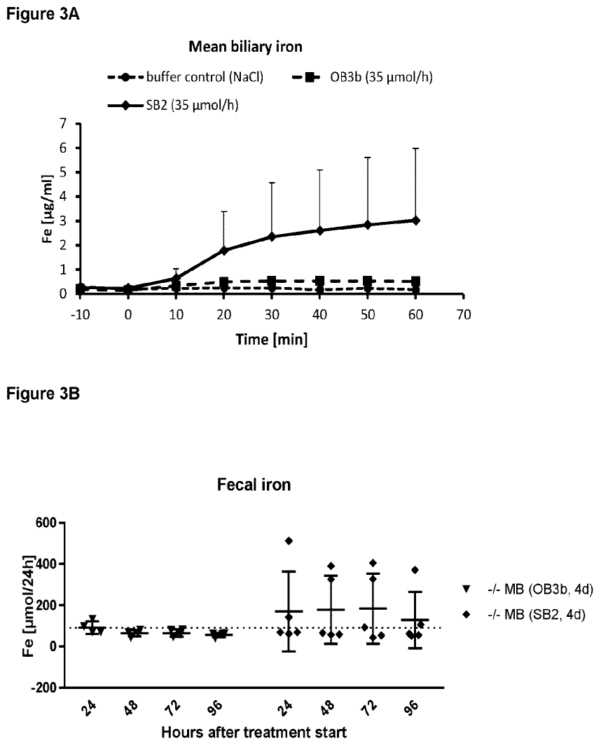

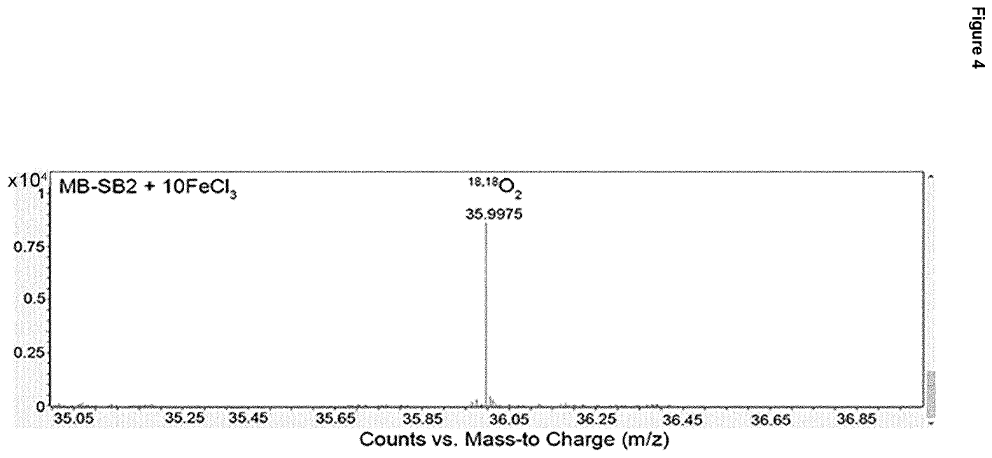

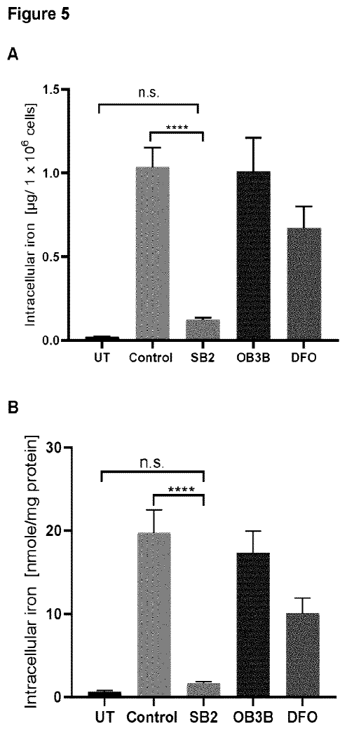

### Example 3: Liver Perfusion Experiments (FIG. 3A)

Cannulated livers from LPP Atp7b^{-/-} rats were perfused for one hour at 37° C. with Krebs-Ringer bicarbonate solution, gassed with 95% O₂ and 5% CO₂, and MB-SB2 or MB-OB3b (35 μmol). During perfusion total bile was collected in 10-minute intervals. Biliary iron concentrations were determined by inductively coupled plasma optical emission spectrometry (ICP-OES) as described (Lichtmannegger et al. J Clin Invest, 2016, 126, 2721-2735).

### Example 4: Fecal Iron Excretion (FIG. 3B)

LPP Atp7b^{-/-} rats were injected intraperitoneally (i.p.) twice a day for four consecutive days with either 110 mg/kg bw MB-SB2 or 150 mg/kg bw MB-OB3b. Rats were housed individually in metabolic cages for 4 days. Feces of each rat was collected in 24-hour periods at 24, 48, 72 and 96 hours after treatment start. Feces was separated from chow residues, dried, homogenized by grinding or milling and digested with concentrated HNO₃ and iron content determined with inductively coupled plasma optical emission spectrometry (ICP-OES).

### Example 5: Water Oxidation (FIG. 4)

Saturated solutions of anhydrous FeCl₃ were prepared in a Coy anaerobic chamber (atmosphere 95% Ar 5% H₂)(Coy Laboratory Products, Ann Arbor, MI, USA). An amount sufficient to create a 0.5 to 10-fold excess of metal was added to 100 μl of between 1 mM and 10 mM of MB-SB2 or MB-OB3b and head space gas samples were collected from the vial. All solutions were made with 97% H₂¹⁸O (Sigma Aldrich, St. Louis, Mo, USA) in 0-0.8 ml brown airtight vials (DWK Life Sciences, Millville, NJ, USA).

Oxidation of 2H₂O to O₂+4H⁺ in reaction mixtures containing a metal and MB-SB2 was determined by monitoring production of ^{18,18}O₂ and H⁺. In oxygen evolution experiments, freeze-dried MB-SB2, MB-OB3b, catalase, as well as anhydrous metal stock solution were prepared in 97% H₂¹⁸O. Reaction mixtures contained 2 mM MB-SB2 or MB-OB3b and 0.5-20 mM metal in a final volume of 100 μl H₂¹⁸O. Reaction mixtures were prepared in 2 ml brown serum vials, sealed with Teflon lined silicon septa. Initial experiments were determined with aluminum foil wrapped vials, but that practice was discontinued once it was clear that identical results were produced regardless if vials were wrapped or not. Generation of ^{18,18}O₂ from H₂¹⁸O was monitored by direct injection (1μl or 2 μl) of head space.

Gas samples were manually injected into an Agilent 7890B GC system (Santa Clara, CA, USA with a 7250 Accurate-Mass Q-TOF GC/MS and a DB5-ms column. Except for the ^{18,18}O₂ injections for standard curves, all injections were 1 μl using gas tight Hamilton syringes. Standard curves were generated with 1 μl, 1.5 μL and 2 μl injections of 97% ^{18,18}O₂ (Sigma Aldrich, St. Louis, Mo, USA). The head space in the vials was sampled before and after the addition of the metals, as was the outside air in the mass spectroscopy as controls. After the standards and controls were injected, the samples were mixed and head space samples were immediately collected, with subsequent samples taken every 30-60 seconds. After several minutes, collection slowed to 1 sample every 2-3 minutes. The quantization of generated ^{18,18}O₂came from an extracted-ion chromatogram set to 35.9978 Da. A small shift in the MS location of the ^{18,18}O₂ was observed on some dates. If a drift in the MS of ^{18,18}O₂ was observed, identity of the peak was verified with 97% ^{18,18}O₂ standard.

1. A methanobactin reducing Fe³⁺ ions to Fe²⁺ ions for use in medicine.

2. The methanobactin for use of claim 1, wherein the primary structure of the methanobactin comprises a structure according to formula (I):

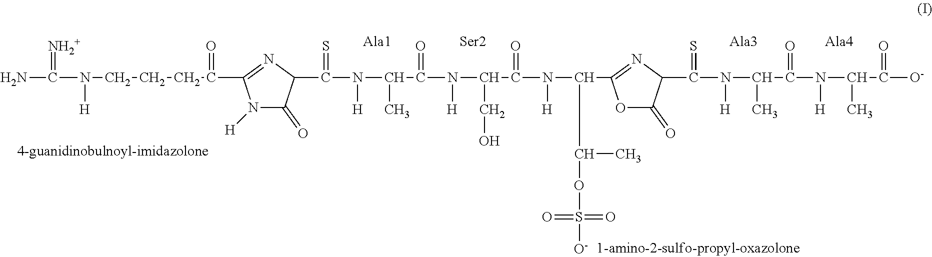 [SMILES: CC(NC(=O)C(C)NC(=S)C1N=C(C(NC(=O)C(CO)NC(=O)C(C)NC(=S)C2N=C(C(=O)CCCNC(N)=[NH2+])NC2=O)C(C)OS(=O)(=O)[O-])OC1=O)C(=O)[O-]]

3. The methanobactin for use of claim 1 or 2, wherein reducing Fe³⁺ to Fe²⁺ comprises the intermediate structure of methanobactin-Fe (II) complex according to formula (II):

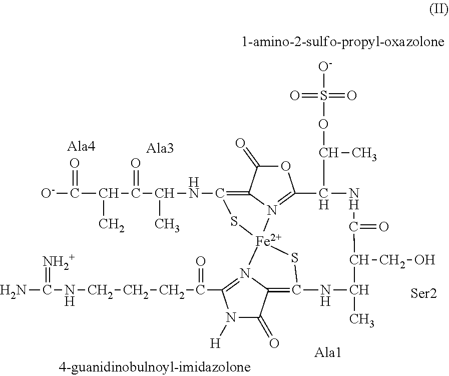

4. The methanobactin for use of claims 1 to 3, wherein the methanobactin is derived from Methylocystis sp. strain SB2.

5. The methanobactin for use of claims 1 to 4, wherein the methanobactin catalyzes the reduction of Fe³⁺ to Fe²⁺ and wherein
a) water is used as reduction agent and molecular oxygen is generated and/or
b) the reduction takes place at a rate of 0.5 to 5, preferably 0.7 to 3, more preferably 1 Fe³⁺ reduced per minute per methanobactin.

6. A pharmaceutical composition for use of in medicine comprising the methanobactin of claims 1 to 5.

7. The methanobactin for use of claims 1 to 5 or the pharmaceutical composition for use of claim 6, in the treatment of an iron-overload disorder, neurodegenerative diseases, iron overload disorder due to red blood cell transfusions, senescence, ageing, ferroptotic cell death, alcoholic liver disease or amyotrophic lateral sclerosis.

8. The methanobactin for use of claim 7 or the pharmaceutical composition for use of claim 7, in the treatment of an iron-overload disorder.

9. The methanobactin for use of claim 7 or 8, or the pharmaceutical composition for use of claim 7 or 8, wherein the iron-overload disorder is caused by a disease which is selected from the group consisting of hereditary hemochromatosis, thalassemia, sickle cell disease, aplastic anemia, myelodysplasia.

10. The methanobactin for use of claim 7 or the pharmaceutical composistion of claim 7, wherein the neurogenerative diseases are selected from the group consisting of Alzheimers disease, Parkinson disease, Dementia, Huntington disease, multiple sclerosis.

11. A process for reduction of Fe³⁺ to Fe²⁺ ex vivo, comprising contacting the methanobactin as defined in claims 1 to 5 with Fe³⁺ ions in a solution optionally in the presence of a suitable reduction agent.

12. The process of claim 11, wherein the methanobactin is a catalyst and a reduction agent is present.

13. The process of claim 11 or 12, wherein
a) the solvent and/or reduction agent is water and/or
b) the reduction takes place at a rate of 0.5 to 5, preferably 0.7 to 3, more preferably 1 Fe³⁺ reduced per minute per methanobactin.

14. The process of claim 13, wherein molecular oxygen is generated.
